(19) United States
(12) Patent Application Publication
(10) Pub. No.: US 2014/0094807 A1
(43) Pub. Date: Apr. 3, 2014

(54) Transpedicular Disk Access System

(71) Applicant: Yosi Weitzman, Tel Aviv (IL)

(72) Inventor: Yosi Weitzman, Tel Aviv (IL)

(21) Appl. No.: 13/633,920

(22) Filed: Oct. 3, 2012

(51) Int. Cl. A61B 17/16

(52) U.S. Cl. CPC A61B 17/1671
USPC 606/80

(57) ABSTRACT

An improved system for treatment of herniated disks is described. Access to a disk is obtained by drilling through a pedicle of an adjacent vertebra, and then curving the passage to pass through the vertebral body into the intervertebral space. The disk is then removed, augmented or repaired from within the internal part of the intervertebral space. Trauma is greatly reduced by this procedure.

### BACKGROUND

Spinal disks can be damaged to produce herniation in several ways. Once herniated, a disk frequently impinges on the nerves associated with the spine, producing pain and often impairing function. Removal of the protruded portions of the disk is usually required.

However, access to regions of disk protrusion is difficult. The disk, which is covered above and below by vertebrae, has an exposed profile that is shielded posteriorly by the spinal cord and the sacral and coccygeal nerves, as well as by the spinous processes. Lateral and anterior access is likewise difficult, because nerves must be avoided, and access is less direct.

Current procedures for reducing herniated or otherwise damaged disks require careful displacement of nerves, especially in the lumbar region, which is the most common site of disk herniation. Even after access to the site of herniation is obtained, reliable stabilization of the herniation is difficult to maintain, because the annulus fibrosus is typically damaged, allowing the disc nucleus to be extruded (or re-extruded) at the site of the defect.

As a result, a common treatment for a damaged disk is to remove the remnants of the disk and fuse together the vertebrae above and below the damaged disk. Often, fixation devices are attached to the spine at loci above and below the damaged disk, to stabilize the spine and to minimize both pain and further local damage to the spine. Pedicles and other readily-accessible points on the spine are used as the points of binding for the various fixation devices, using screws and other type of fasteners. See, for example, Ross (U.S. Pat. No. 7,306,605), which describes devices spanning two or more disks for stabilization.

Another common treatment of the spine is “vertebroplasty”, the repair or stabilization of damaged vertebrae which cause spinal pain. Because access to the vertebrae is difficult, even when access to the disk is not required, there are some similarities in techniques and approaches between procedures for disk repair and procedures for vertebral repair. However, surgical device sets and the details of surgical procedures are not the same in these conditions. Examples of vertebral stabilization or repair include, for example, the above cited Ross, U.S. Pat. No. 7,306,605; U.S. 2010/0298832 to Lau et al; U.S. 2006/0009846 to Trieu et al; and U.S. Pat. No. 4,805,602 to Puno et al. These procedures often eliminate or reduce the pain arising from a ruptured disk, generally by removal of the disk and fusion of the adjacent vertebrae. However, forces arising from bending of the spine are now increased in the disks above and below the site of disk removal. Often, adjoining disks fail due to the increased stress. Treatment becomes even more difficult, and the prognosis in such a situation is not favorable.

There is a distinct literature on devices for removal of damaged disks, with or without fusion of the vertebrae. These include patents such as Johnson et al, U.S. Pat. No. 5,695,513; Schaller, U.S. Pat. No. 7,670,374; Schaller et al, U.S. 2010/0161060; and Michelson U.S. Pat. No. 7,914,530.

Johnson et al, U.S. Pat. No. 5,695,513, teaches the advantages in surgery of a helically wound cable of strands of nitinol or equivalent, for making a drive cable that withstands sharp bends during operation without breaking.

Schaller, U.S. Pat. No. 7,670,374, props up damaged spinal components (e.g., FIG. 2) with a wire coil “injected” into a site within a vertebra (FIG. 2, 13-14), or within a spinal disk (FIG. 26C) which is then covered by a tube. After withdrawal of the coil, the tube can be filled with a stabilizing material. Similar intra-vertebral tubes are found in FIGS. 30-58 in vertebrae, and in FIGS. 59-78 in disk repair; vertebral replacement is shown in FIGS. 79-84. The emphasis is on distraction of the disk or vertebra at the treatment site to restore previous distances among components.

Schaller et al, U.S. 2010/0161060, which is similar in overall subject matter, enters the disc through the side (FIG. 4-6), thereby incurring most of the problems of older disk treatment methods using that route; as noted above, penetration of a disk from any of its sides is often a source of trauma due to damage to nerves and blood vessels.

Michelson, U.S. Pat. No. 7,914,530, stabilizes a vertebral segment by drilling out the disk and adjacent vertebral tissue, and then implanting a permanent fixation device in the resulting space. Minimization or avoidance of trauma to nerves is not discussed. This is, in effect, a form of spinal fusion, with its advantages and disadvantages.

Bilsky (Neurosurgical Focus 2000:9(4); “Transpedicular Approach to Thoracic Disk Herniation”; available at www.medscape.com) describes a direct, slanting approach to the disk space via the pedicle. Bone cuts are required, and a close approach to the spinal cord is used to access the herniated region of the disk (see FIG. 1). Only a limited set of indications are described as being suitable for treatment by this route; effects of the bone cuts on long-term stability are not described.

An improved method for disk repair and stabilization is clearly needed. The present invention provides a method for accessing selected lesions of a spinal disk via a route that is both transpedicular and transvertebral, so that repair or replacement of a disk can be preformed percutaneously, with complete repair of the access route to preserve vertebral integrity, and with minimal trauma to the nerves and vessels of the spine. In particular, direct lateral penetration of the disk is avoided.

### SUMMARY OF THE INVENTION

An improved route of access to spinal disks, especially lumbar disks, is described. The route of access is both transpedicular and transvertebral, avoiding lateral penetration of the disk. The invention further comprises an improved method of access to the spinal disk, and the use of the method for treatment of conditions affecting the spine.

In one aspect of the invention, a spinal disk is accessed by creating a passage which enters the body of a vertebra adjacent to said disk via a pedicle of said vertebra. After penetrating through the pedicle into the vertebral body, the continuation of the passage is bent sufficiently, for example by about 90 degrees but without specific limitation, so that the passage proceeds through the vertebral body, penetrates the vertebra at the disk, and arrives at the vertebral surface of the disk. By selection of pedicle (left or right), and by control of angle within the vertebral body, the point or region of access to the disk can be specifically targeted and controlled, allowing precision of treatment.

Once the disk has been accessed, many techniques can be applied to the disk, including complete or partial removal of a damaged disk, and complete or partial replacement or reinforcement of a disk with materials or devices for improving spinal flexibility, or for relieving pain associated with loss of intervertebral spacing.

After completion of the procedure, the passage that was made to access the disk is preferably filled with bone substitute e.g. cement or with other suitable materials, to achieve prevention of flow through said passage, or strengthening of the body of the vertebra. In addition, other materials can be deposited in the access passage, or in or near the inter-annular disk space, for therapeutic effect. Such filling of the passage with appropriate materials and techniques allows further therapies if needed in the future, e.g. using transpedicular screws.

In particular, the system of the invention comprises a device for transpedicular treatment of spinal disks. The device comprises a suite of tools that fit together to be operable to access a disk. The suite components typically comprise at least a penetrator to penetrate into the cancellous bone of a pedicle of a vertebra adjacent to the disk to be treated; a bendable sheath; a steerable burr; and a sleeve locatable over said sheath. The device may further comprise materials for lining all or part of the passageway leading from the external surface of the pedicle to a location adjacent to a disk. The device may further comprise means for removal of disk material, or means for delivery of bone graft material to fill channels in bone opened during said procedure.

In one aspect, the device further comprises a spring-loaded guide to allow lateral positioning of a steerable burr or other rotary tissue remover within the lumen of the device. Said guide may enable an eccentric positioning of a bone cutting burr, to allow the burr to cut a larger diameter path through the bone of the vertebra than the diameter of the tube or sheath carrying said burr. The steerable burr may be positioned on a flexible shaft.

In another aspect, the invention comprises a method for removing disk tissue, the method comprising a first step of passing a penetrator, such as a needle (closed or hollow), a drill, or other device, through a pedicule and into a vertebral body to provide a passageway; a second step of passing a bendable sheath through said penetrator or said passageway, while bending said sheath as required; a third step of passing a powered burr into said sheath; and further steps, concurrently or in alternation, comprising one or more of rotating said burr, advancing said shaft and bending said sheath. The bendable sheath may be partially withdrawn and then re-introduced through the previous passage, as required to create a passageway having the total required bending.

In particular, the method may comprise the steps of providing a passageway extending through a pedicle into a vertebra; passing a bendable sheath into said passageway; passing a powered burrinto said sheath; and concurrently or in alternation, performing one or more of rotating said burr, advancing said shaft, and bending said sheath.

In another aspect, a method for removing disk material comprises tissue removal tools adapted for trans-pedicular access to the disk via a vertebra. The tools may comprise a spring-loaded guide to allow lateral positioning of a steerable burr, and further may comprise an eccentric positioning of a bone cutting burr so that said burr, when projected from a carrying tube, can cut a larger diameter path through the bone of the vertebra than the diameter of the tube carrying said burr. The method may further comprise the placement of said steerable burr on a flexible shaft.

In another aspect, the invention comprises a method of relieving back pain, the method comprising providing trans-pedicular access to the interior of a vertebral body that is adjacent to a disk to be treated; passage of a bendable sheath into said vertebral body; passage through said bendable sheath of a rotatable burr for removing bone within a vertebra; and bending said sheath to create a passage extending to said disk.

### BRIEF DESCRIPTION OF THE FIGURES

FIGS. 1-9 show a method for creation and closure of a passage through a vertebra to access a spinal disk, comprising a series of steps. FIG. 1 shows the entry of a needle into the cancellous bone of a pedicule. FIG. 2 shows the passage of a flexible, steerable burr for bone ablation through the needle. FIG. 3 shows the burr being bent. FIG. 4 shows the advance of the burr along the axis of the disk. FIG. 5 shows the contact of the burr with the endplate of the disk. FIG. 6 shows the entry of the burr into the intervertebral disk space. FIG. 7 shows a sleeve that has been slid over the burr, after which the burr is retracted. FIG. 8 shows the use of the sleeve to advance tools for removal of pieces of the disk nucleus. In FIG. 9, the instruments have been removed and the opening created is filled with bone graft material.

FIG. 10 shows an alternative method of providing a passage for access via a pedicle and the associated vertebra to the disk space.

### DETAILED DESCRIPTION OF THE INVENTION

The invention comprises a method for repair or amelioration of defects in the intervertebral space and spinal disk, and a system for accomplishing such repair. The novel path to the intervertebral space provided by the invention allows the execution of a variety of procedures in the intervertebral space without significant compromise of the integrity of the spine.

Vertebral pedicules are widely used in the art as sites for attachment of bone screws and the like for fixation of the spine at a particular joint, using a plate or other fixation device. The pedicle is a desirable site for attachment because it can be reached percutaneously while avoiding nerves and major blood vessels. Such procedures are used for alleviation of intractable pain, but typically lead to further progression of deterioration at adjacent joints up or down the spine from the fixated site. The temporary relief leads to long term destruction of vertebrae.

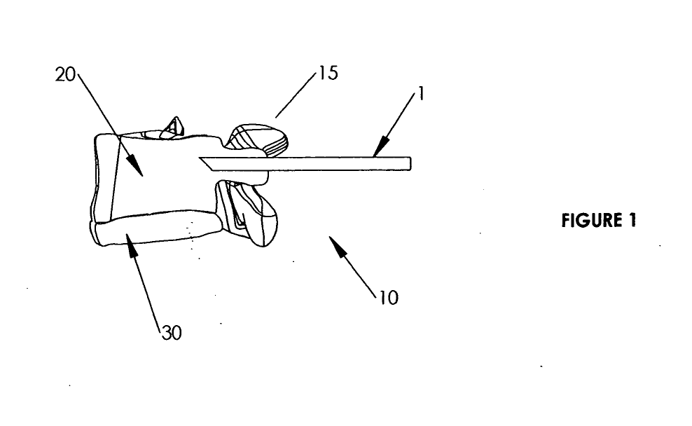

The improved method and access route to the spinal disc provided by the invention is described with reference to the figures. FIGS. 1-9 show drawings of the steps in the procedure.

FIG. 1 is a cross-section of a vertebra 10 and spinal disk 30. It shows the initial entry of a needle 1 into a pedicle 15 of the vertebra 10, having body 20. The needle is depicted as hollow, but an initial entry could be made with a solid needle or by a drill (not illustrated), which could then be replaced by a hollow needle or cannula 1, or simply omitted, for the following steps.

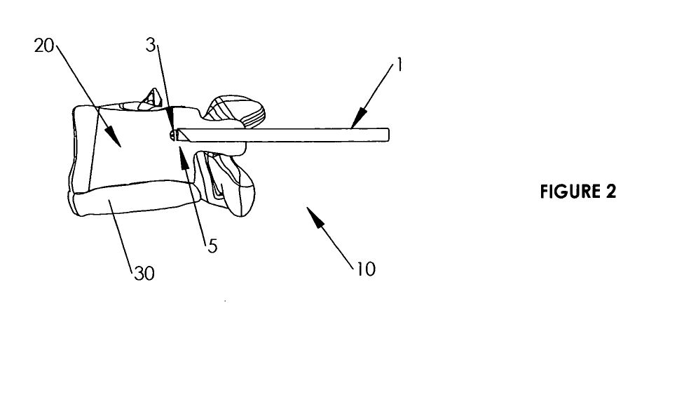

In FIG. 2, a rotary burr 3 has been advanced through cannula 1 and has been used to cut an entry segment into the cancellous bone of the vertebral body 20. The rotary burr 3 is carried on the tip of a bendable steering cannula 5. The bendable steering cannula 5 may, in a preferred embodiment, be a somewhat flexible hollow tube with means available for bending said tube in a controlled way. The steering cannula 5 may also be of other designs, including designs having partially fenestrated walls, or being at least partially formed by braiding, or otherwise distinct from a flexible hollow tube but having the same functionality.

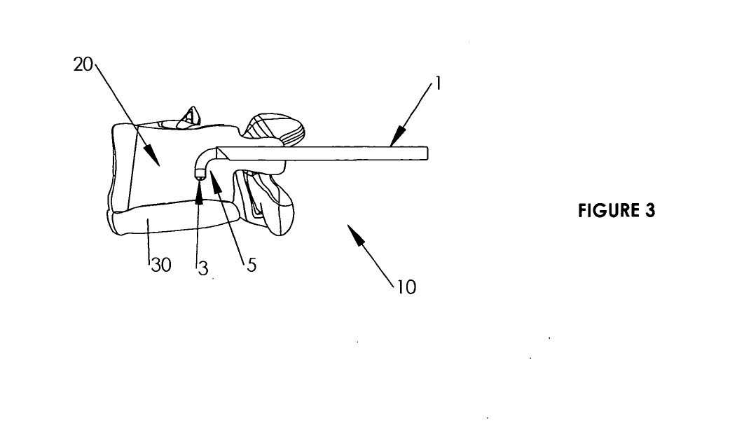

FIG. 3 shows the next step in the process, in which the bendable steering cannula 5 is gradually bent, while burr 3 is sufficiently active to cut a path through the cancellous bone to allow the bend. The actual path of movement of the tip will typically create extra space (not shown) through which the bendable steering cannula 5 will pass.

The bending can be made by any convenient means. In a preferred embodiment, the bending is made by pull wires running in the walls of the cannula 1 or in hollow tubes attached to the cannula 1 (not illustrated). In another embodiment, the cannula 1 can have inherent curvature, which is straightened by the insertion of a rigid tube (not illustrated) into the cannula; bending occurs spontaneously when the cannula is moved forwards on the rigid tube.

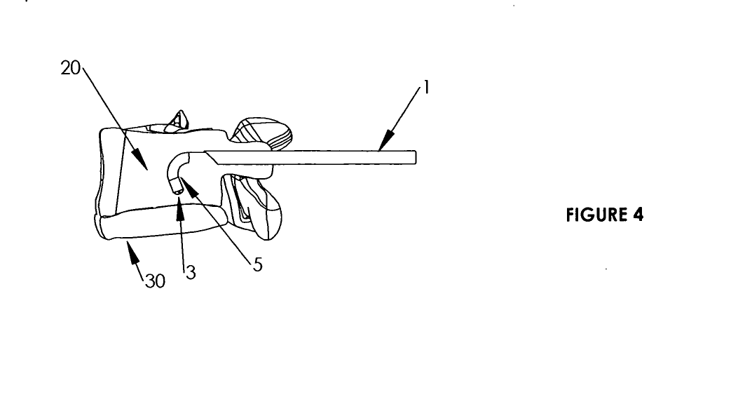

FIG. 4 shows the burr 3 and steering cannula 5 being further advanced through the vertebral body. The bend in this illustration is somewhat greater than a right angle (90 degrees), to allow treatment at a site on the posterior edge of the disk. The bend angle could also be less than 90 degrees to allow treatment of the anterior region of the disk. The burr could also be advanced to the right or the left of the center vertical plane of the disc if desired (not illustrated.) Advancement to one side or the other of the disk can be easier to achieve if the entry point is on the pedicle on the opposite side of the spine from the desired disk entry point such that the required bending angle and radius are larger. “Lateral” bending can be accomplished by the addition of a second set of bending wires, or by an initial entry beyond the space created by needle 1, or equivalent, in which a plane of entry is not parallel to the axis of the spine.

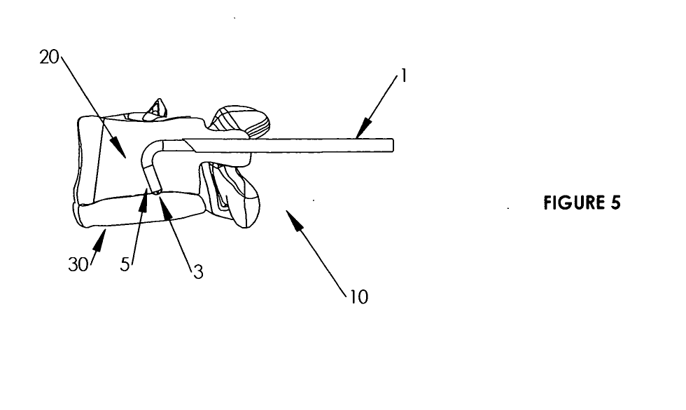

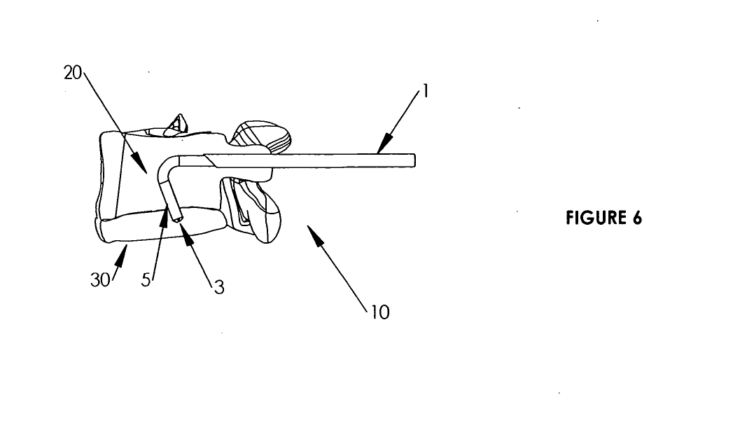

FIG. 5 shows the further advance of the burr 3 to the surface of the spinal disk 30, and FIG. 6 shows the penetration of the disk 30 by the burr 3 and its steering cannula 5.

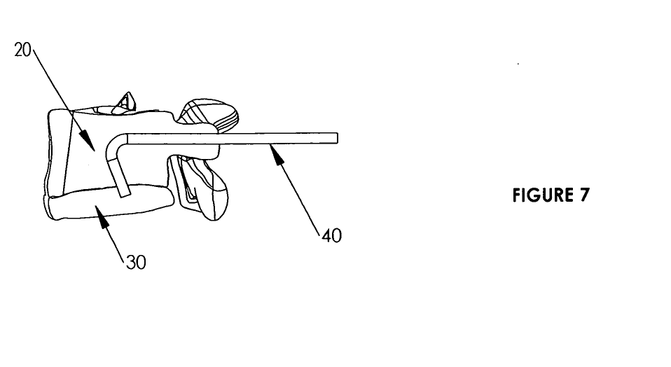

The disk 30 is typically soft and flexible compared to the surrounding bone, and does not cut well with the same type of rotary burr 3 used to create the passage through the bone. As shown in FIG. 7, a sleeve 40 can be advanced over the needle 1 and burr 3, to the site of treatment within the disk 30. Then the needle, steering device and burr can be removed.

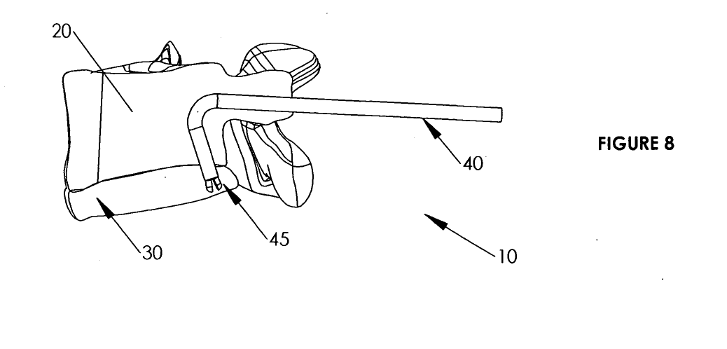

Next, as shown in FIG. 8, the sleeve 40 can guide instruments 45 into the disk 30 for removal of pieces of the disk nucleus or periphery to allow relief of pressure, or to create an empty space to be filled with material of an appropriate modulus. A grasping/cutting device is shown. Alternatively, a modified burr (not illustrated), adapted for the less rigid tissue of the disk, can be passed through the sleeve 40, optionally initially though tube 1. Materials for filling a space created in disk 30 can then be passed to the site in the disk through sleeve 40. Alternatively, if the burr 3 can readily be removed from the disk area without removal of cannula 5, then cannula 5 can be used for passage of materials out of or into the disk 30.

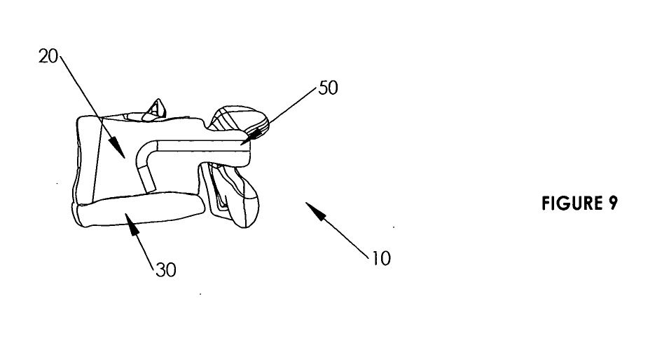

After any treatment or replacement of the disk is finished, the sleeve or equivalent is removed. The passageway formed in the vertebra during the procedure is preferably filled with bone graft or other material 50, as shown in FIG. 9, for greater post-operative stability. The filling material may also comprise active ingredients, such as antibiotics, reinforcing materials, or other materials for treating the spine.

Either pedicle of the spinal disk can be used for entry into the vertebral body, and approach to the disk can be either upwards or downwards, i.e. through the vertebral body below the disk to be treated, or the vertebral body above the disk. (Only the latter is illustrated.)

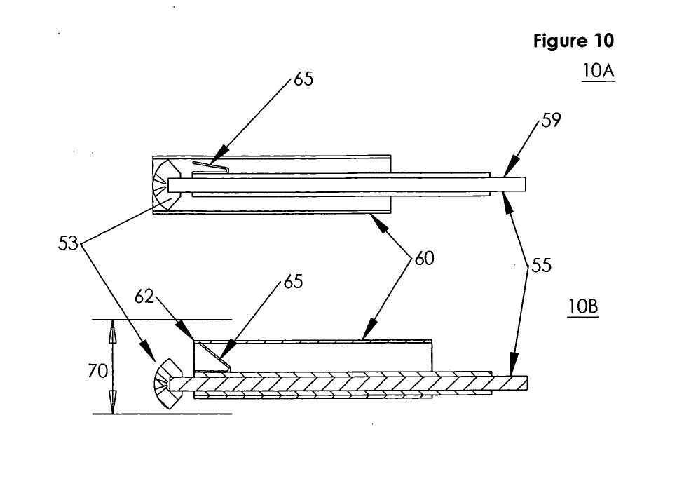

FIG. 10 shows an alternative embodiment of a system, which is designed to create a curved passage through a vertebra. The curved passage has a diameter 70 which is larger than the diameter of any of the components. The system has a rotary burr 53, a drive shaft 55 for driving the burr 53, a positioning tube 59 for the drive shaft 55, and an outer or steering tube 60. The system further comprises a spring clip 65 mounted on positioning tube 59.

The embodiment of system FIGS. 10A, 10B shows a burr 53 mounted on a shaft 55 being passed through outer tube 60. A spring clip 65 is mounted on one side of positioning tube 59, and presses the burr 53 against the opposite side of outer tube 60. Preferably, the burr is not operated while it is within tube 60. The system as shown in FIG. 10A is somewhat similar to the system as previously described. However, in FIG. 10A the spring clip 65 pushes the burr 53 against the opposite side of tube 60, potentially increasing the wear on the burr and tube. Hence, the burr is typically not operated while within tube 60.

The system of FIG. 10 further comprising means to allow positioning of said burr so that said burr can remove material beyond the outer boundary of said bendable sheath. Said means may comprise a spring clip 65 attached to a carrier tube positioned within said bendable sheath.

FIG. 10B (lower panel) shows the effect of pushing shaft 55 further to the left, so that burr 53 is pushed beyond the terminal end 62 of tube 60, and now the burr can be operated to remove tissue. However, the spring clip 65 now biases the shaft 55 to the opposite side of tube 60, so that the burr 53 extends laterally beyond the diameter of the outer tube 60. The torque generated by the attack of the burr 53 on the surrounding tissue (not shown) tends to cause the positioning tube 59 to rotate within the outer tube 60, given appropriate selection of the force of spring clip 65. This causes the outer edge of the burr 53 to extend beyond the edge of tube 60. Tube 60 can be rotated, partially or completely, such that the diameter 70 of the passageway created by the action of the burr 53 can be significantly greater than the diameter of the outer tube 60. This extra width of the tube 70 that is created in the tissue provides clearance between the instrument outer tube 60 and the surrounding tissue, and thereby makes it easier to navigate the instrument through bends, such as those shown in FIG. 1-9.

After creating a passageway to a desired site, the burr is retracted into tube 60 by pulling on shaft 55. Then a sleeve may be installed over tube 60, using it as a guide as described above, and the burr and ancillary tubes can be removed through the sleeve. The passageway that was created may likewise be filled with bone substitute or other material at the conclusion of the procedure, to preserve the strength of the vertebral body.

Further means for navigating inside the vertebral body, especially in locating the endplate or the disc surface, may be provided by one or more devices. Examples include:
an audio pickup capable of identifying a change in burr operation noise;
a sensor capable of identifying a change in the tissue mechanical rigidity;
means of measuring the torque required for rotating the burr;
means of measuring the magnetic field around the tip;
means of measuring the electric conductivity of the tissue;
means of measuring the thermal conductivity of the tissue;
means of measuring the tissue removal rate.

The device may further comprise means for navigating the tip to a specific location in the target disc, e.g. a herniation. This may be accomplished by any of a variety of useful means, including in particular by injecting a radio-opaque fluid through the channel.

### Use of the Device

A herniated disk, in which some of the disk material has been extruded through a lesion in the side of the disk, can be treated as follows. A needle is inserted into a pedicle and bone is removed or pushed aside to create a first passage. The penetrating needle is removed and a hollow needle is used to stabilize the passage through the cancellous bone. A bendable steering cannula is inserted into the hollow needle. The cannula carries a burr, which may be a largely enclosed burr as shown in FIG. 2 or a burr that can be pushed out beyond the bendable steering cannula as shown in FIG. 10. The burr is activated and pushed distally if needed, and a generally cylindrical passage is made in the bone beyond the first passage. By application of bending torque to the tip of the cannula, a bent passage is created and extended.

When the cannula reaches the target site inside the vertebra, and penetrates into the disk space, the burr is removed. Then, other instruments may be used to remove tissue within the disk. The cannula can also be used to deliver disk replacement or enhancement materials into the disk space. These may include sealing materials to minimize further herniation of the disk, and other materials, synthetic or natural, to restore the disk's natural shock-absorbing and flexibility functions. If required, more than one type of material may be deposited in the disk. For example, a first sealing material could be delivered to a lesion in the annulus of the disk, and then a force-distributing material could fill the inside of the disk. Then the disk and any replacement material could be sealed to prevent leakage.

Finally, the cannula and any accessory devices are removed. The passage that was created is preferably filled with bone cement or a functional equivalent to strengthen the vertebra and prevent outflow of disk materials. Other materials may be delivered to the inside or the vicinity of the disk before or during the sealing of the passage.

This improved system for repair of herniated disks has the potential to provide a significant improvement of spinal disk function in a single procedure that will have a short recovery period.

1. A system for transpedicular treatment of spinal disks, the system comprising:
a penetrator to penetrate into cancellous bone of a pedicle of a vertebra adjacent to the disk to be treated, and to create a first passage within said cancellous bone;
a bendable sheath which passes through said first passage;
means for bending said sheath;
a burr capable of passing through said sheath, and means for driving it;
and a sleeve locatable over said sheath;
wherein said treatment is conducted without direct lateral penetration of said disk.

2. The system of claim 1 further comprising removal tools for disk tissue passable through at least one of said bendable sheath and said sleeve.

3. The system of claim 1 further comprising means for delivery of material to close channels that were created in bone during said procedure.

4. The system of claim 1 further comprising the placement of said burr on a flexible shaft.

5. The system of claim 4 further comprising positioning means to allow positioning of said burr so that said burr can remove material beyond the outer boundary of said bendable sheath, wherein said positioning means comprise a spring clip attached to a carrier tube positioned within said bendable sheath.

6. The system of claim 5, further comprising spring-loaded guides that allow said burr to make paths having a wider diameter than the diameter of the sheath.

7. The system of claim 1, characterized in having a rotary burr, a drive shaft, a positioning tube, and a steering device, and further comprising a spring clip mounted on said positioning tube.

8. The system of claim 7, wherein said spring clip positions said rotary burr, when positioned distally of a tube and rotated around said tube's perimeter and within it, sufficiently laterally of said tube that a channel is cut which is larger in diameter than said cutting burr.

9. The system of claim 1, further comprising means for navigating inside the vertebral body, wherein the means are provided by one or more devices selected from:
a pickup capable of identifying a change in burring noise;
a sensor capable of identifying a change in the tissue mechanical rigidity;
means of measuring the torque required for rotating the burr;
means of measuring the magnetic field around the tip;
means of measuring the electric conductivity of the tissue;
means of measuring the thermal conductivity of the tissue;
means of injecting a radio-opaque fluid through a channel in the body; and
means of measuring the tissue removal rate.

10. A method for removing disk tissue, the method comprising the steps of:
providing a passageway extending through a pedicle into a vertebra body;
passing a bendable sheath into said passageway;
passing a powered burr into said sheath;
and concurrently or in alternation, performing at least one of rotating said burr, advancing said sheath, and bending said sheath.

11. The method of claim 10 further comprising removal tools for disk tissue, wherein said tools are passable through at least one of said bendable sheath and a sleeve riding in or on said sheath.

12. The method of claim 10 further comprising means for delivery of material to close channels that were created in bone during the passage of the removal tools.

13. The method of claim 10 further comprising the placement of said burr on a flexible shaft.

14. The method of claim 13 further comprising means to allow positioning of said burr so that said burr can remove material beyond the outer boundary of said bendable sheath.

15. The method of claim 14 wherein said means comprises a spring clip attached to a carrier tube that is positioned within said bendable sheath.

16. The method of claim 15, further comprising spring-loaded guides that allow said burr to make paths having a wider diameter than the diameter of the sheath.

17. The method of claim 10, characterized in providing a rotary burr, a drive shaft, a positioning tube, and a steering device, and further comprising providing a spring clip mounted on said positioning tube.

18. A method for treatment of a spinal disk, the method comprising making a first channel through a vertebra to the surface of said disk by use of a burr; retracting said burr; and using the resulting channel for passage of instruments for the specific treatment of said disk.

19. The method of claim 18, further including one or more of
complete or partial removal of a damaged disk;
complete or partial replacement of the disk;
reinforcement of a disk with materials or devices for avoiding future herniation or rupture improving spinal flexibility; and
relieving pain associated with loss of intervertebral spacing.

20. The method of claim 18, wherein after completion of the procedure, the passage that was made to access the disk is filled with materials suitable to achieve one or more of prevention of flow through said passage, strengthening of the body of the vertebra, and deposition of materials for therapeutic effect.
